(19) United States
(12) Patent Application Publication
(10) Pub. No.: US 2018/0071000 A1
(43) Pub. Date: Mar. 15, 2018

(54) TELESCOPING FIXATION DEVICES AND METHODS OF USE

(71) Applicant: Arthrex, Inc., Naples, FL (US)

(72) Inventors: Trung Pham, Santa Rosa, CA (US); Carl J. Basamania, Edmonds, WA (US); Stephen Riddle McDaniel, San Rafael, CA (US); Daryll Leonard Charles Fletcher, Santa Rosa, CA (US); Alexander Carlo Buscaglia, Chicago, IL (US); Kyle Edward Lappin, Lake Zurich, IL (US)

(21) Appl. No.: 15/702,843

(22) Filed: Sep. 13, 2017

Related U.S. Application Data
(60) Provisional application No. 62/393,946, filed on Sep. 13, 2016.

(51) Int. Cl. A61B 17/86

(52) U.S. Cl. CPC A61B 17/8685; A61B 17/8625; A61B 2017/00991; A61B 17/8615; A61B 17/864

(57) ABSTRACT

The present application relates to fixation devices, and more particularly to telescoping fixation devices and methods of use. The fixation devices described herein can include an elongate member. The fixation devices described herein can include a barrel, wherein the elongate member and the barrel are designed to telescope along a longitudinal axis and wherein the barrel is designed to transmit torque to the elongate member. The fixation devices described herein can include an inner screw designed be inserted into the barrel and engage the elongate member.

### BACKGROUND

### Field

The present application relates to fixation devices, and more particularly to telescoping fixation devices and methods of use.

### Description of the Related Art

Bone fractures are a common medical condition both in the young and old segments of the population. As one example, sports and work-related accidents account for a significant number of bone fractures seen in emergency rooms among all age groups. Further, the acceptance and ubiquitous application of passive restraint systems in automobiles has created greater numbers of non-life threatening fractures. In the past, a person that might expire from a serious automobile accident now survives with multiple traumas and resultant fractures. With an increasingly aging population, osteoporotic fractures have increased. Osteoporosis and osteoarthritis are among the most common conditions to affect the musculoskeletal system, as well as frequent causes of locomotor pain and disability. The National Osteoporosis Foundation estimates that as many as 44 million Americans are affected by osteoporosis and low bone mass, leading to fractures in more than 300,000 people over the age of 65.

Osteotomy is the surgical cutting of bone. The bone can be cut to shorten, lengthen, or to change the alignment of the bone. As one example, a bone can be cut to correct the alignment of a bone that has healed incorrectly after a fracture. Examples include osteotomies of the hip to improve alignment of the acetabulum or femoral head and osteotomies of the knee to improve alignment of the knee. Other osteotomies are known in the medical field.

Arthrodesis is the surgical immobilization of adjacent bones by fusion. The joint can be fused by introduction of a material into the joint, such as the introduction of a bone graft created from bone or synthetic bone substitutes. In some surgical methods, the joint can be fused by an implant to facilitate bony fusion.

### SUMMARY

The fixation devices and related methods have several features, no single one of which is solely responsible for its desirable attributes. Without limiting the scope as expressed by the claims that follow, the more prominent features of the systems and methods will now be discussed briefly. After considering this discussion, and particularly after reading the section entitled “Detailed Description” one will understand how the features of the systems and methods provide several advantages over other devices.

One of the advantages of embodiments of the fixation devices described herein include that the fixation devices are simple, intuitive devices. The fixation devices advantageously have few parts, making the fixation devices robust and cost-effective to manufacture.

The fixation devices can be used in a variety of surgeries, such as minimally invasive surgery. For instance, in various embodiments, the fixation devices can be used in all surgeries, and in particular embodiments, the fixation device is favorable in the repair of fractures, osteotomies, and arthrodesis.

In some embodiments, a fixation device is provided with an elongate member; a barrel, wherein the elongate member and the barrel are configured to telescope along a longitudinal axis, wherein the barrel is configured to transmit torque to the elongate member; and an inner screw configured to be inserted into the barrel and engage the elongate member.

In some embodiments, the inner screw is configured to translate the barrel toward the elongate member. In some embodiments, at least a portion of the inner screw is threaded. In some embodiments, at least a portion of the elongate member is threaded. In some embodiments, the elongate member comprises a lumen. In some embodiments, at least a portion of the lumen of the elongate member is threaded. In some embodiments, the barrel comprises a lumen. In some embodiments, at least a portion of the elongate member is configured to translate within the lumen of the barrel. In some embodiments, the fixation device is cannulated. The fixation device of can include one or more engagement members configured to engage a surface of the elongate member to transmit torque from the barrel to the elongate member. In some embodiments, the one or more engagement members comprise one or more cylindrical pins. In some embodiments, the surface comprises a flat surface. In some embodiments, the one or more engagement members are configured to extend through the barrel in a direction perpendicular to the longitudinal axis. In some embodiments, the surface comprises a groove. In some embodiments, the one or more engagement members are configured to extend through the barrel in a direction parallel to the longitudinal axis. In some embodiments, the one or more engagement members is a non-round internal surface of the barrel.

In some embodiments, a method of manufacturing a fixation device is provided. The method can include the step of providing a barrel comprising a lumen and a retention structure. The method can include the step of inserting an elongate member through the lumen of the barrel. The method can include the step of inserting an engagement member in the retention structure, wherein the barrel is configured to transmit torque to the elongate member after inserting the engagement member.

In some embodiments, the elongate member and the barrel are configured to telescope along a longitudinal axis after inserting the engagement member. In some embodiments, the elongate member and the barrel are configured to translate a fixed distance after inserting the engagement member. In some embodiments, the engagement member comprises a cylindrical pin. In some embodiments, the retention structure comprises a cylindrical slot. In some embodiments, the engagement member translates along a flat surface of the elongate member. In some embodiments, the engagement member is configured to extend through the barrel in a direction perpendicular to the longitudinal axis. In some embodiments, the engagement member translates along a groove of the elongate member. In some embodiments, the engagement member is configured to extend through the barrel in a direction parallel to the longitudinal axis.

In some embodiments, a method of use of a fixation device is provided. The method can include the step of inserting a fixation device into a bone, the fixation device comprising an elongate member and a barrel, wherein the elongate member and the barrel are coupled. The method can include the step of rotating the barrel to telescope the elongate member. The method can include the step of inserting an inner screw into the fixation device.

In some embodiments, inserting the fixation device into bone comprises inserting the fixation device over a guide pin. In some embodiments, inserting the fixation device into bone comprises inserting the fixation device without the inner screw inserted into the fixation device. In some embodiments, inserting the fixation device into bone comprises rotating the barrel. In some embodiments, inserting the fixation device into bone comprises inserting the fixation device in a shortened position. In some embodiments, the elongate member and the barrel are rotationally coupled. In some embodiments, the elongate member and the barrel are coupled to allow translation between the elongate member and the barrel. The method can include the step of contacting a head of the fixation device with the bone. The method can include the step of rotating the barrel to telescope the elongate member after contacting a head of the fixation device with the bone. In some embodiments, rotating the barrel to telescope the elongate member further comprises rotating the barrel in place. In some embodiments, rotating the barrel to telescope the elongate member further comprises rotating the barrel without translating the barrel. The method can include the step of inserting the inner screw into the barrel after rotating the barrel to telescope the elongate member. The method can include the step of rotating the inner screw to compress the barrel toward the elongate member. The method can include the step of retaining the inner screw within the barrel.

In some embodiments, a fixation device is provided. The fixation device can include an elongate member; a barrel, wherein the elongate member and the barrel are configured to telescope along a longitudinal axis, wherein the barrel is configured to transmit torque to the elongate member; and a nut configured to be inserted into the barrel and engage the elongate member.

In some embodiments, the nut is configured to translate the barrel toward the elongate member. In some embodiments, at least a portion of the nut is threaded. In some embodiments, at least a portion of the elongate member is threaded. In some embodiments, the nut comprises a lumen. In some embodiments, at least a portion of the lumen of the nut is threaded. In some embodiments, the barrel comprises a lumen. In some embodiments, at least a portion of the elongate member is configured to translate within the lumen of the barrel. In some embodiments, the fixation device is cannulated. In some embodiments, one or more engagement members configured to engage a surface of the elongate member to transmit torque from the barrel to the elongate member. In some embodiments, the one or more engagement members is a non-round internal surface of the barrel.

In some embodiments, a method of use of a fixation device is provided. The method can include the step of inserting a fixation device into a bone, the fixation device comprising an elongate member and a barrel, wherein the elongate member and the barrel are coupled. The method can include the step of rotating the barrel to telescope the elongate member. The method can include the step of inserting a nut into the barrel.

In some embodiments, inserting the fixation device into bone comprises inserting the fixation device over a guide pin. In some embodiments, inserting the fixation device into bone comprises inserting the fixation device without the nut inserted into the barrel. In some embodiments, inserting the fixation device into bone comprises rotating the barrel. In some embodiments, inserting the fixation device into bone comprises inserting the fixation device in a shortened position. In some embodiments, the elongate member and the barrel are rotationally coupled. In some embodiments, the elongate member and the barrel are coupled to allow translation between the elongate member and the barrel. The method can include the step of contacting a head of the fixation device with the bone. The method can include the step of rotating the barrel to telescope the elongate member after contacting a head of the fixation device with the bone. In some embodiments, rotating the barrel to telescope the elongate member further comprises rotating the barrel in place. In some embodiments, rotating the barrel to telescope the elongate member further comprises rotating the barrel without translating the barrel. The method can include the step of inserting the nut into the barrel after rotating the barrel to telescope the elongate member. The method can include the step of rotating the nut to compress the barrel toward the elongate member. The method can include the step of retaining the nut within the barrel.

### BRIEF DESCRIPTION OF THE DRAWINGS

These and other features, aspects, and advantages of the invention disclosed herein are described below with reference to the drawings of preferred embodiments, which are intended to illustrate and not to limit the invention. Additionally, from figure to figure, the same reference numerals have been used to designate the same components of an illustrated embodiment. The following is a brief description of each of the drawings.

FIG. 1 is a perspective view of an embodiment of a fixation device;

FIG. 2 is a perspective view of the fixation device of FIG. 1 in another position;

FIG. 3 is a cross-sectional view of the fixation device of FIG. 1;

FIG. 4 is a cross-sectional view of the fixation device of FIG. 3 with an inner screw;

FIG. 5 is a cross-sectional view of the fixation device of FIG. 2;

FIG. 6 is a perspective view of the fixation device of FIG. 1 with the inner screw;

FIG. 7 is a perspective view of the engagement members and the elongate member of FIG. 1;

FIG. 8 is a perspective view of the engagement members and the barrel of FIG. 1;

FIG. 9 is a perspective view of an embodiment of a fixation device;

FIG. 10 is a view of the elongate member of the fixation device of FIG. 9;

FIG. 11 is a view of the barrel of the fixation device of FIG. 9;

FIG. 12 is a perspective view of an embodiment of a fixation device;

FIG. 13 is a view of the elongate member of the fixation device of FIG. 12;

FIG. 14 is a view of the barrel of the fixation device of FIG. 12;

FIG. 15 is an exploded view of the fixation device of FIG. 12;

FIG. 16 is a perspective view of an embodiment of a fixation device;

FIG. 17 is a view of the elongate member of the fixation device of FIG. 16;

FIG. 18 is a view of the nut of the fixation device of FIG. 16;

FIG. 19 is a view of components of the fixation device of FIG. 16.

### DETAILED DESCRIPTION

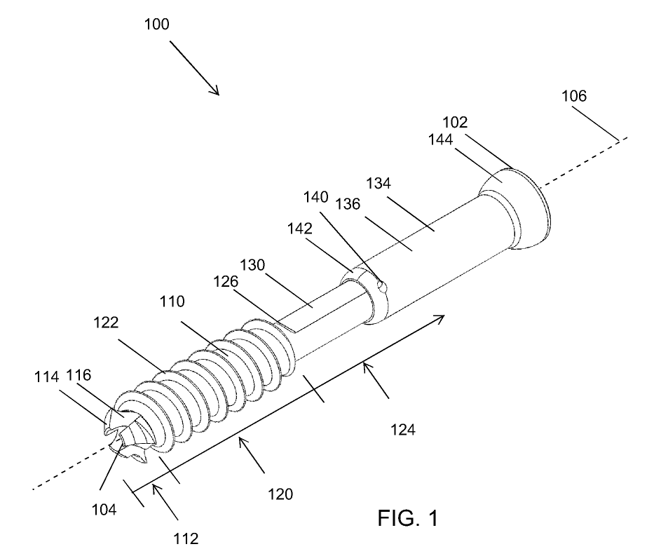

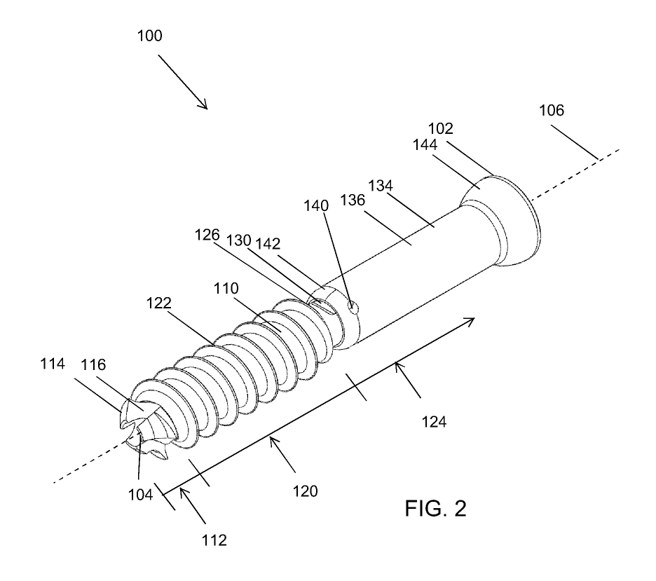

FIGS. 1 and 2 are perspective views of the fixation device 100. The fixation device 100 includes a proximal end 102 (near the user) and a distal end 104 (further from the user). The proximal end 102 and the distal end 104 refer to the position of an end of the fixation device 100 relative to the remainder of the fixation device 100 or relative to the opposing end as it appears in the drawings. The proximal end 102 can refer to the end that is manipulated by the user. The distal end 104 can refer to the end that is inserted and advanced within the bone. The use of proximal and distal can change in another context, for instance in an anatomical context in which proximal and distal are relative to the patient, or where the entry point is distal from the user.

The fixation device 100 includes a longitudinal axis 106 extending from the proximal end 102 to the distal end 104. In the illustrated embodiment, the longitudinal axis 106 is straight or substantially straight. In some embodiments, the longitudinal axis 106 includes one or more bends or curves.

The fixation device 100 can include an elongate member 110. The elongate member 110 can extend from the distal end 104 toward the proximal end 102. The elongate member 110 can include a first section 112. The first section 112 can be considered the cutting section or the insertion section. The first section 112 can be located near the distal end 104. In the illustrated embodiment, the first section 112 comprises one or more projections 114. The illustrated embodiment includes three projections 114, but other embodiments are contemplated (e.g., one projection, two projections, three projections, four projections, five projections, six projections, etc.). Each projection 114 can include a cutting face 116. The cutting face 116 can be planar or substantially planar. The cutting face 116 can include one or more edges. The edges can be sharpened to facilitate the cutting of bone.

The fixation device 100 can be rotated to be driven into bone. As the fixation device 100 is rotated, the cutting face 116 and/or the associated edges of the cutting face 116 are designed to cut the bone. The design of the cutting face 116 can allow bone fragments to be moved away from the cutting face 116 during rotation.

The elongate member 110 can include a second section 120. The second section 120 can be considered the purchase section or grip section. In the illustrated embodiment, the second section 120 is located proximally to the first section 112. The second section 120 can include a thread 122. The thread 122 can be a helical structure as shown. The thread 122 can be an external thread. The thread 122 can be designed for penetration of bone, for instance by selecting one or more dimension such as lead, pitch and start. In the illustrated embodiment, the thread 122 can be right-handed, but in some applications, left-handed thread can be used. The thread 122 can be a coarse thread to increase purchase or grip with the bone. In some embodiments, the second section 120 includes two or more threads 122, for instance a double helical thread, two starts, or threads having different dimensions such as pitch, major diameter, or minor diameter. The largest diameter of the second section 120 can be greater than the largest diameter of the first section 112.

The fixation device 100 can be rotated to be driven into bone. As the fixation device 100 is rotated, the thread 122 is designed to cut the bone. The design of the thread 122 can allow bone fragments to be moved between the thread 122 during rotation. In some embodiments, the fixation device 100 includes only the first section 112. In some embodiments, the fixation device 100 includes only the second section 120. The fixation device 100 can include one or more structures to engage bone including one or more flanges, one or more ridges, one or more expandable grippers, one or more spikes, one or more deformable structures, one or more threads, one or more projections, one or more cutting faces, etc. The fixation device 100 can include one or more structures designed to cut bone. The fixation device 100 can include one or more structures designed to engage bone. The fixation device 100 can include one or more structures designed to lodge the fixation device 100 within a canal of a bone. The fixation device 100 can include one or more structures designed to lodge the fixation device 100 within a bored hole in bone.

The elongate member 110 can include a third section 124. The third section 124 can be considered the translation section. In the illustrated embodiment, the third section 124 is located proximally to the first section 112. In the illustrated embodiment, the third section 124 is located proximally to the second section 120. The largest diameter of the third section 124 can be less than the largest diameter of the second section 120.

The third section 124 can include a shoulder 126. The third section 124 can include one or more driving sections 130. In the illustrated embodiment, each driving section 130 includes a flat surface including a length and a width. The length can be measured along the longitudinal axis 106. The width can be measured in a direction orthogonal to the longitudinal axis 106. Each driving section 130 can be considered a flat. The shoulder 126 can separate the second section 120 and the driving sections 130.

In the illustrated embodiment, the third section 124 includes two driving sections 130. The two driving sections 130 are located on opposite sides of the third section 124. The two driving sections 130 are diametrically opposed. The two driving sections 130 are separated by 180 degrees. Other embodiments are contemplated (e.g., one driving section, two driving sections, three driving sections, four driving sections, five driving sections, six driving sections, seven driving sections, eight driving sections, etc.). The one or more driving sections 130 are designed to transmit torque from the proximal end 102 to the distal end 104 of the fixation device 100.

The third section 124 can have a major diameter. The major diameter can be orthogonal the longitudinal axis 106. The major diameter can be the largest diameter of the third section 124. The major diameter can be the diameter of the shoulder 126. The third section 124 can have a minor diameter. The minor diameter can be measured between opposing driving sections 130. The minor diameter is less than the major diameter. The ratio of the minor diameter to the major diameter can be any one of the following: 0.5:2, 0.75:2, 1:2, 1.25:2, 1.5:2, 1.75:2, etc. In the illustrated embodiment, the largest diameter of the second section 120 can be greater than the largest diameter of the third section 124.

The fixation device 100 can include a barrel 134. The barrel 134 can be cylindrical or generally cylindrical. The barrel 134 can include an exterior surface 136. The exterior surface 136 of the barrel 134 can be cylindrical or generally cylindrical. The exterior surface 136 of the barrel 134 can be smooth or substantially smooth. The diameter of the exterior surface 136 can be approximately equal to the largest diameter of the elongate member 110. The diameter of the exterior surface 136 of the barrel 134 can be approximately equal to the largest diameter of the second section 120 of the elongate member 110. The diameter of the exterior surface 136 of the barrel 134 can be equal or approximately equal to the diameter of the thread 122. The diameter of the exterior surface 136 of the barrel 134 can be less than the diameter of the third section 124 of the elongate member 110.

The barrel 134 can include one or more retention structures 140. Each retention structure 140 is designed to retain an engagement member, as described herein. The number of retention structures 140 can correspond to the number of engagement members. The number of retention structures 140 can correspond to the number of driving sections 130. While two retention structure are shown, other configurations are contemplated (e.g., one retention structure, two retention structures, three retention structures, four retention structures, five retention structures, six retention structures, seven retention structures, eight retention structures, etc.). Each retention structure 140 can include a slot. The slot can be designed to match the cross-sectional shape of the engagement member. In the illustrated embodiment, the retention structures 140 include two cylindrical slots. The one or more retention structures 140 can include an edge or other interlocking feature to prevent the release of the engagement member.

The barrel 134 can include a taper 142. The taper 142 can be located at the interface between the barrel 134 and the elongate member 110. The taper 142 can reduce the diameter of the barrel 134 to facilitate insertion of the barrel 134 in the bone. The taper 142 can be located toward the distal end 104 of the fixation device 100, when the fixation device 100 is assembled.

The barrel 134 can include a head 144. The head 144 can be located near the proximal end 102, when the fixation device 100 is assembled. The largest diameter of the head 144 can be greater than the diameter of the exterior surface 136 of the barrel 134. The largest diameter of the head 144 can be greater than the largest diameter of the elongate member 110. The largest diameter of the head 144 can be greater than the largest diameter of the second section 120 of the elongate member 110. The largest diameter of the head 144 can be greater than the diameter of the thread 122.

The fixation device 100 can include a first position. The first position can be the position of the greatest total length of the fixation device 100. The total length can be measured along the longitudinal axis 106. The elongate member 110 can extend over a portion of the total length of the fixation device 100 in the first position. In the first position, the elongate member 110 can extend approximately 50% of the total length, approximately 60% of the total length, approximately 70% of the total length, approximately 80% of the total length, approximately 90% of the total length, more than 50% of the total length, more than 60% of the total length, more than 70% of the total length, more than 80% of the total length, more than 90% of the total length, etc.

The fixation device 100 can include a second position. The second position can be the position of the shortest total length of the fixation device 100. The elongate member 110 can extend over a portion of the total length of the fixation device 100 in the second position. In the second position the elongate member 110 can extend approximately 50% of the total length, approximately 60% of the total length, approximately 70% of the total length, approximately 80% of the total length, approximately 90% of the total length, more than 50% of the total length, more than 60% of the total length, more than 70% of the total length, more than 80% of the total length, more than 90% of the total length, etc. FIG. 1 shows the fixation device 100 lengthened toward the first position. FIG. 2 shows the fixation device 100 shortened toward the second position.

The fixation device 100 can telescope. The fixation device 100 can comprise concentric components that can slide or be caused to slide into itself, so that the fixation device 100 shortens. In the illustrated embodiment, the elongate member 110 and the barrel 134 can be concentric components. In the illustrated embodiment, the elongate member 110 can slide or be caused to slide into the barrel 134. In the illustrated embodiment, the barrel 134 can slide or be caused to slide along the elongate member 110. The fixation device 100 can lengthen by translating toward the first position. The fixation device 100 can shorten by translating toward the second position.

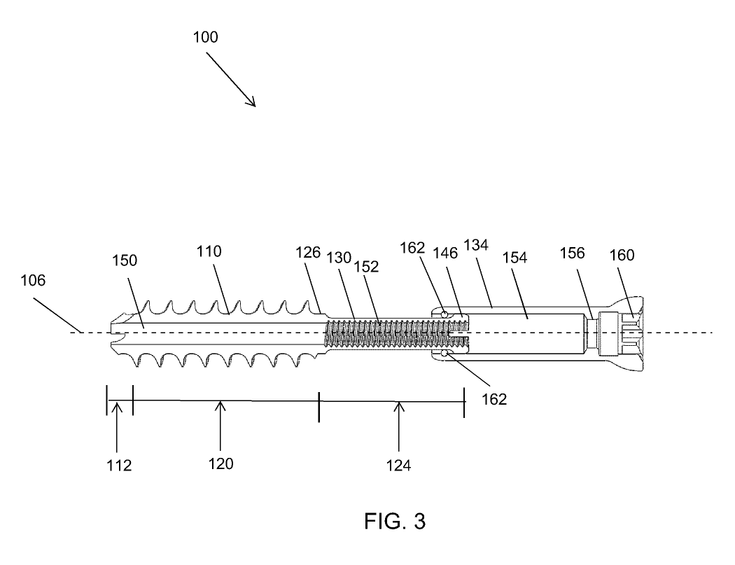

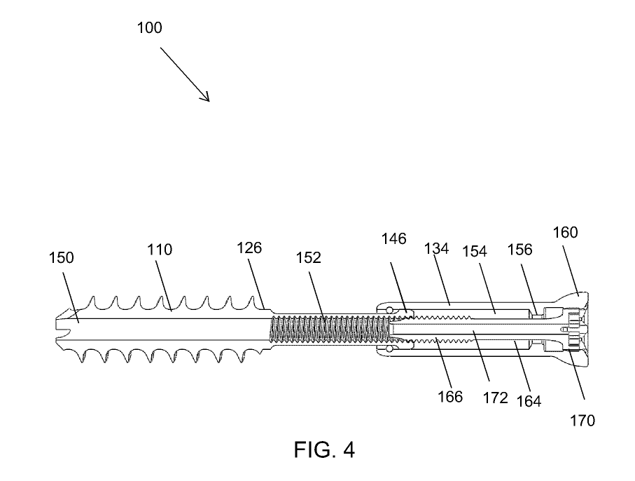

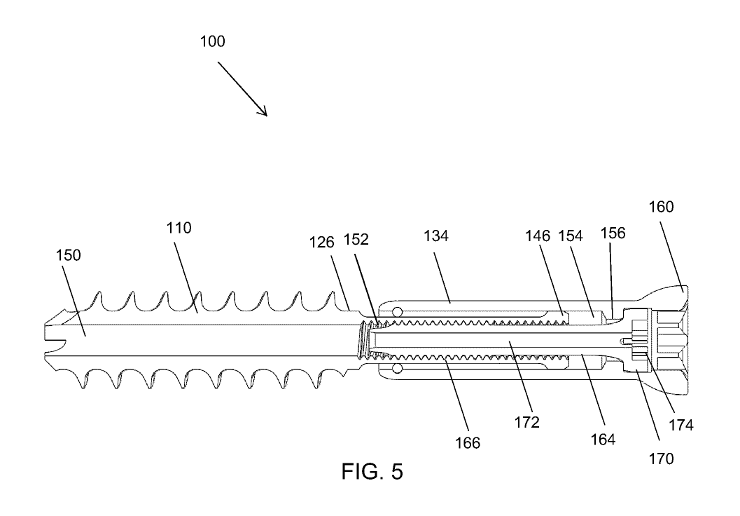

FIGS. 3-5 are cross-sectional views of the fixation device 100. FIGS. 3 and 4 show the fixation device 100 of FIG. 1. FIG. 5 shows the fixation device 100 of FIG. 2.

FIG. 3 shows the first section 110, the second section 120, and the third section 124 of the elongate member 110. The third section 124 can include a head 146. In some embodiments, the head 146 has the same diameter as the shoulder 126. The largest diameter of the head 146 can be greater than the minor diameter of the third section 124. The largest diameter of the head 146 can be equal or approximately equal to the major diameter of the third section 124.

The elongate member 110 can be cannulated. The elongate member 110 can include a lumen 150. The lumen 150 can extend along the longitudinal axis 106. The lumen 150 can include a threaded portion 152. The threaded portion 152 can be internal threads. The threaded portion 152 can extend the length of the third section 124 or a portion thereof. In some embodiments, the threaded portion 152 can extend one or more of the following portions of the elongate member 110: the first section 112, the second section 120, the third section 124, the shoulder 126 of the third section 124, the driving portions 130 of the third section 124, and the head 146 of the third section 124.

The barrel 134 can be cannulated. The barrel 134 can include a lumen 154. The lumen 154 of the barrel 134 can extend along the longitudinal axis 106. In the illustrated embodiment, at least a portion of the elongate member 110 is designed to slide within the barrel 134. The head 146 of the elongate member 110 is designed to slide within the lumen 154 of the barrel 134. The third section 124 of the elongate member 110, or a portion thereof, is designed to slide within the lumen 154 of the barrel 134.

The lumen 154 of the barrel 134 can include additional features. The lumen 154 of the barrel 134 can include a stop 156. The stop 156 can be designed to limit distal movement of an inner screw, as described herein. The stop 156 can be designed to be a surface upon which a compressive force of the inner screw is applied. In some embodiments, the stop 156 can be designed to limit proximal movement of the head 146 of the elongate member 110 within the lumen 154 of the barrel 134. The fixation device 100 can be in the second position when the head 146 of the elongate member 110 abuts the stop 156.

The lumen 154 of the barrel 134 can include a socket 160. The socket 160 can include a plurality of flats, such as a torx socket. The socket 160 can be designed to engage a tool (not shown) for transmitting torque to the barrel 134. Other configurations of sockets are contemplated including hex, 12-point flange, tri-wing, double-square, triple square, polydrive, spline drive, double hex, bristol, and pentalobe, etc.

The fixation device 100 includes one or more engagement members 162. In the illustrated embodiment, the fixation device 100 includes two engagement members 162. The number of engagement members 162 can correspond to the number of driving sections 130. In the illustrated embodiment, each driving section 130 includes an engagement member 162. The one or more engagement members 162 are designed to interact with the one or more driving sections 130.

The two engagement members 162 are located on opposite sides of the fixation device 100. The two engagement members 162 are diametrically opposed. The engagement members 162 are separated by 180 degrees. Other embodiments are contemplated (e.g., one engagement member, two engagement members, three engagement members, four engagement members, five engagement members, six engagement members, seven engagement members, eight engagement members etc.). The one or more engagement members 162 are designed to transmit torque from the proximal end 102 to the distal end 104 of the fixation device 100.

In the illustrated embodiment the one or more engagement members 162 have a circular cross-section. The one or more engagement members 162 can be cylindrical or generally cylindrical. The one or more engagement members 162 can be spherical or generally spherical. Other cross-sectional and three-dimensional shapes are contemplated (e.g., square, rectangle, polygon, triangle, elliptical, irregular, regular, conical, etc.).

As shown in FIG. 1, the barrel 134 can include one or more retention structures 140. The one or more engagement members 162 can be designed to be retained within the one or more retention structures 140. In some embodiments, each retention structure 140 retains one engagement member 162. The one or more engagement members 162 can be designed to roll within the one or more retention structures 140. The one or more engagement members 162 can be designed to rotate within the one or more retention structures 140. The one or more engagement members 162 can be immobile relative to the one or more retention structures 140.

The one or more engagement members 162 can extend into the lumen 154 of the barrel 134. The one or more engagement members 162 can reduce the diameter of the lumen 154 of the barrel 134. The one or more engagement members 162 can reduce the diameter of the lumen 154 of the barrel 134 to approximately the minor diameter of the third section 124. The one or more engagement members 162 can engage the third section 124 of the elongate member 110. The one or more engagement members 162 can engage the one or more driving sections 130.

The one or more engagement members 162 can function as a stop. The one or more engagement members 162 can be designed to limit distal movement of the head 146 of the elongate member 110 within the lumen 154 of the barrel 134. The fixation device 100 can be in the first position when the one or more engagement members 162 abut the head 146 of the elongate member 110. The one or more engagement members 162 can be designed to limit proximal movement of the shoulder 126 of the elongate member 110 within the lumen 154 of the barrel 134. The fixation device 100 can be in the second position when the one or more engagement members 162 abut the shoulder 126 of the elongate member 110.

In the illustrated embodiment, the translation between the elongate member 110 and the barrel 134 is limited. In some embodiments, the translation is limited by two or more components abutting. In some embodiments, the translation is limited by the elongate member 110 abutting the one or more engagement members 162, as described herein. In some embodiments, the translation is limited by the head 146 abutting the one or more engagement members 162, as described herein. In some embodiments, the translation is limited by the shoulder 126 abutting the one or more engagement members 162, as described herein.

In the illustrated embodiment, the elongate member 110 and the barrel 134 are rotationally coupled. In some embodiments, the elongate member 110 and the barrel 134 are rotationally coupled by two or more components abutting. In some embodiments, the elongate member 110 and the barrel 134 are rotationally coupled by the elongate member 110 abutting the one or more engagement members 162, as described herein. In some embodiments, the elongate member 110 and the barrel 134 are rotationally coupled by the one or more driving sections 130 abutting the one or more engagement members 162, as described herein.

Referring to FIGS. 4 and 5, the fixation device can include an inner screw 164. The inner screw 164 can be designed to be inserted into the barrel 134. The inner screw 164 can be designed to be inserted into the elongate member 110. The inner screw 164 can be designed to be inserted into the head 146 of the elongate member 110. The inner screw 164 can have a length sufficient to engage the elongate member 110 when the fixation device 100 is assembled.

The inner screw 164 can include a thread 166. The thread 166 can be an external thread. The thread 166 can be designed to engage the threaded portion 152 of the lumen 150 of the elongate member 110. In the illustrated embodiment, the thread 166 can be right-handed, but in some applications, left-handed thread can be used. The thread 166 of the inner screw 164 can be the same handed thread as the thread 122 of the elongate member 110 (e.g., both right-handed thread). The thread 166 of the inner screw 164 can be the opposite handed thread as the thread 122 of the elongate member 110 (e.g., one right handed thread, one left handed thread).

The inner screw 164 can include a head 170. The head 170 of the inner screw 164 can be designed to be inserted within the socket 160 of the barrel 134. The head 170 of the inner screw 164 can be designed to abut the stop 156. The stop 156 is designed to limit distal movement of the inner screw 164 within the barrel 134. In particular, the stop 156 is designed to limit distal movement of the head 170 of the inner screw 164 within the lumen 154 of the barrel 134. The inner screw 164 can have a length sufficient to allow the thread 166 of the inner screw 164 to engage the threaded portion 154 of the elongate member 110 when the head 170 of the inner screw 164 abuts the stop 156.

The head 170 can apply compressive force to the fixation device 100. The head 170 can apply a compressive force on the barrel 134 to move the barrel 134 toward the distal end 104. The head 170 can apply compressive force to the stop 156. The head 170 can apply compressive force to rigidly couple the elongate member 110 and the barrel 134. The inner screw 164 can be rotated to transmit a compressive force. In some embodiments, the inner screw 164 can be rotated in only one direction. In some embodiments, the inner screw 164 can be rotated in both directions. In some embodiments, the inner screw 164 can be rotated to shorten the fixation device 100. In some embodiments, the inner screw 164 can be rotated to lengthen the fixation device 100. As one example, rotation of the inner screw 164 in one direction can move the proximal end 102 toward the distal end 104. As another example, rotation of the inner screw 164 in the opposite direction can move the proximal end 102 away from the distal end 104.

The fixation device 100 is designed to permit translation between the barrel 134 and the elongate member 110 when the inner screw 164 is rotated. As one example, the barrel 134 can translate toward the elongate member 110 when the inner screw 164 is rotated. As one example, the one or more engagement members 162 translate relative to the one or more driving sections 130 when the inner screw 164 is rotated. Each engagement member 162 translates along a driving section 130 when the inner screw 164 is rotated.

In some embodiments, the elongate member 110 and the barrel 134 do not rotate when the inner screw 164 is rotated. The fixation device 100 is designed to limit rotation of the barrel 134 relative to the elongate member 110 when the inner screw 164 is rotated.

The head 170 can include one or more features to prevent back-out. The head 170 can include one or more features to prevent loosening of the inner screw 164. The head 170 can include one or more features to prevent loosening of the fixation device 100. The head 170 can splay outward once inserted into the barrel 134. The head 170 can form an interference fit or frictional fit with the barrel 134. The head 170 can prevent withdrawal of the screw 164 once the screw 164 is inserted into the barrel 134.

The inner screw 164 can be cannulated. The inner screw 164 can include a lumen 172. The lumen 172 of the inner screw 164 can extend along the longitudinal axis 106.

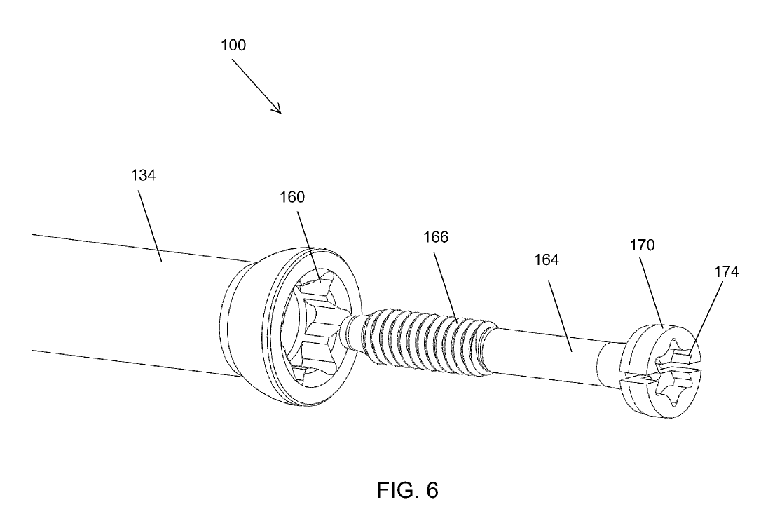

FIG. 6 shows a perspective view of the inner screw 164 and the socket 160 of the barrel 134. FIG. 6 shows the body of the inner screw 164 including the threads 166. The head 170 of the inner screw 164 can include a socket 174. The socket 174 can include a plurality of flats, such as a torx socket. The socket 174 is designed to engage a tool for transmitting torque to the inner screw. Other configurations of sockets are contemplated, as described herein.

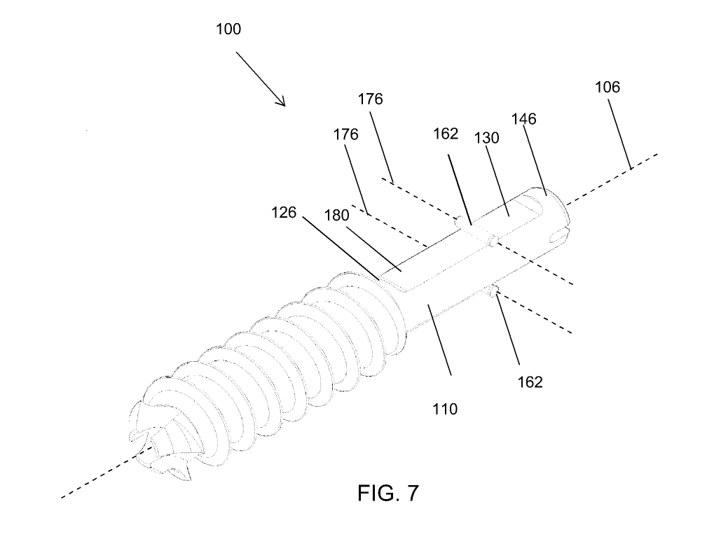

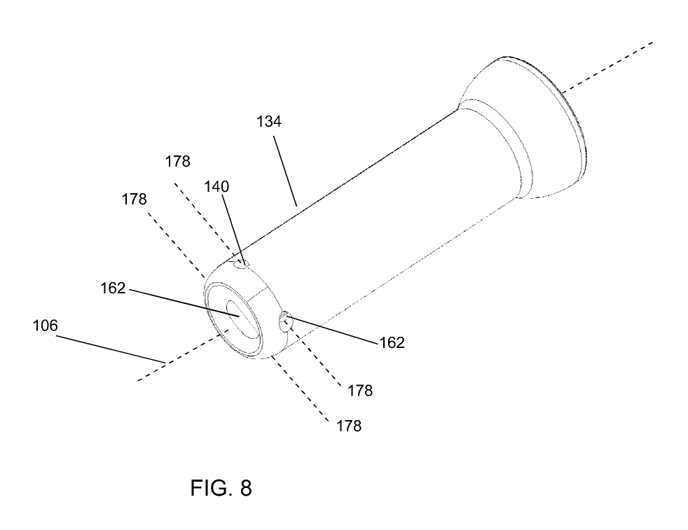

FIG. 7 shows a perspective view of the one or more engagement members 162. The barrel 134 has been removed from the view of FIG. 7. FIG. 8 shows a perspective view of the one or more engagement members 162 installed in the barrel 134. The elongate member 110 has been removed from the view of FIG. 8.

As described herein, the fixation device 100 can include one or more engagement members 162. In the illustrated embodiment, the fixation device 100 includes two engagement members 162. In the illustrated embodiment, the one or more engagement members 162 are cylindrical pins. The one or more engagement members 162 can include a circular cross-section. The one or more engagement members 162 can be cylindrical or generally cylindrical. The one or more engagement members 162 can include an elongated structure. In some embodiments, each of the one or more engagement members 162 has the same shape. In some embodiments, each of the one or more engagement members 162 has the size. In some embodiments, two of the one or more engagement members 162 are different (e.g., different shape, size, cross-sectional shape, three-dimensional shape, material, etc.).

The one or more engagement members 162 can include a longitudinal axis 176. The longitudinal axis 176 can be parallel to a flat surface 180 of the driving section 130. The longitudinal axis 176 can be perpendicular to the longitudinal axis 106. The one or more engagement members 162 can be parallel. The shoulder 126 and the head 146 of the elongate member 100 are shown relative to the one or more engagement members 162.

Referring to FIG. 8, the barrel 134 can include one or more retention structures 140. The one or more retention structures 140 can generally match the cross-sectional shape of the one or more engagement members 162. In the illustrated embodiment, the one or more retention structures 140 of the barrel 134 are cylindrical slots. The one or more retention structures 140 can include a through axis 178 which is perpendicular to the longitudinal axis 106. In some embodiments, the one or more engagement members 162 are press-fit into the one or more retention structures 140 of the barrel 134.

The one or more engagement members 162 can be considered anti-rotation features. As described herein, the one or more engagement members 162 can be disposed in one or more retention structures 140 of the barrel 134. The one or more engagement members 162 can engage the surface of the driving section 130 across the width of the driving section 130. The one or more engagement members 162 can engage the driving section 130 in a direction perpendicular to the longitudinal axis 106.

The one or more engagement members 162 can prevent the elongate member 110 from rotating relative to the barrel 134. The one or more engagement members 162 can be consider torque-transmitting features. The one or more engagement members 162 allow torque to be transmitted from the barrel 134 to the elongate member 110. The one or more engagement members 162 can be considered a rotational link. The one or more engagement members 162 engage the driving sections 130 of the elongate member 110 such that the elongate member 110 and the barrel 134 rotate together.

As described herein, the one or more engagement members 162 can function as a stop to limit translation in the proximal and/or distal direction. Referring to FIG. 7, the one or more engagement members 162 can be designed to limit distal movement of the head 146 of the elongate member 110. The head 146 can abut the one or more engagement members 162 to limit distal movement of the elongate member 110. The one or more engagement members 162 can be designed to limit proximal movement of the shoulder 126 of the elongate member 110. The shoulder 126 can abut the one or more engagement members 162 to limit proximal movement of the elongate member 110. The translation of the elongate member 110 can be limited by the length of the driving sections 130. The length can be measured along the longitudinal axis 106.

The one or more engagement members 162 can prevent separation of the barrel 134 and the elongate member 110. The one or more engagement members 162 cannot extend past the head 146 of the elongate member 110. The one or more engagement members 162 cannot extend past the shoulder 126 of the elongate member 110. The one or more engagement members 162 prevent the barrel 134 from separating from the elongate member 110 when the one or more engagement members 162 are disposed within the one or more retention structures 140 of the barrel 134.

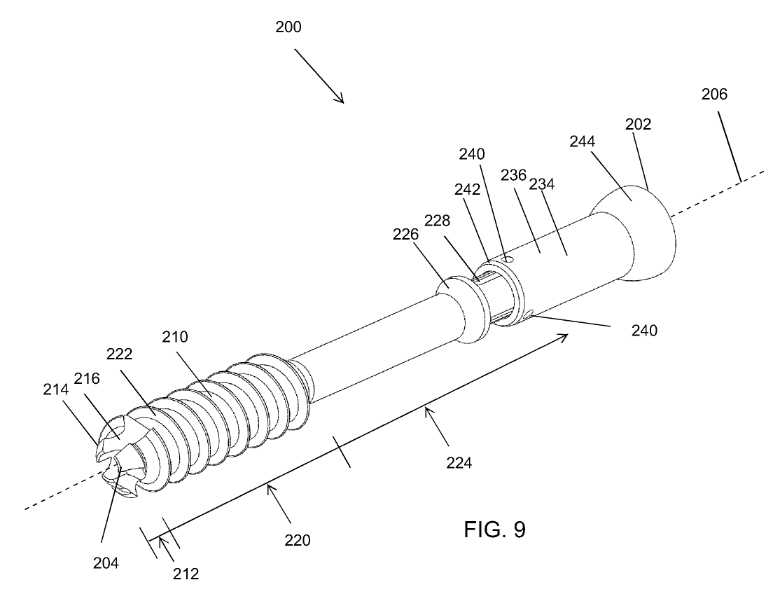

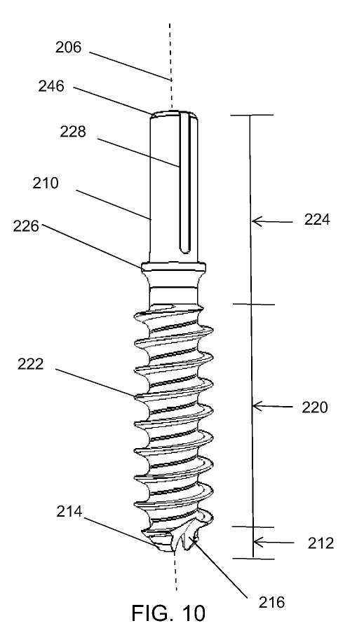

FIG. 9 shows an embodiment of a fixation device 200. The fixation device 200 can include any of the features described herein in relation to the fixation device 100. The fixation device 200 can include a proximal end 202, a distal end 204, and a longitudinal axis 206. The fixation device 200 can include an elongate member 210. FIG. 10 is a view of the elongate member 210 of the fixation device 200. The elongate member 210 can include a first section 212 with one or more projections 214 and one or more cutting faces 216. The elongate member 210 can include a second section 220 with a thread 222.

The elongate member 210 can include a third section 224. The third section 224 can include one or more driving sections 228. The driving sections 228 can include a groove. The driving sections 228 can be narrower than the driving sections 130 of FIGS. 1-5. The driving section 228 can extend in a direction parallel to the longitudinal axis 206. The driving sections 228 can include a curved cross-sectional shape. The elongate member 210 can include a shoulder 226. The driving section 228 can extend along any portion of the third section 224. The elongate member 210 can include a head 246. The driving section 228 can extend along the head 246 of the elongate member 210. The elongate member 210 can include a lumen 250 (not shown) and a threaded portion 252 (not shown) designed to interact with an inner screw 264 (not shown). The elongate member 210 can have similar features to the elongate member 110. The inner screw 264 can have similar features to the inner screw 164. The shoulder 226 can be enlarged relative to the barrel 234, as shown in FIG. 9. The fixation device 200 can include one or more elongate portions between the section 220 and the shoulder 226.

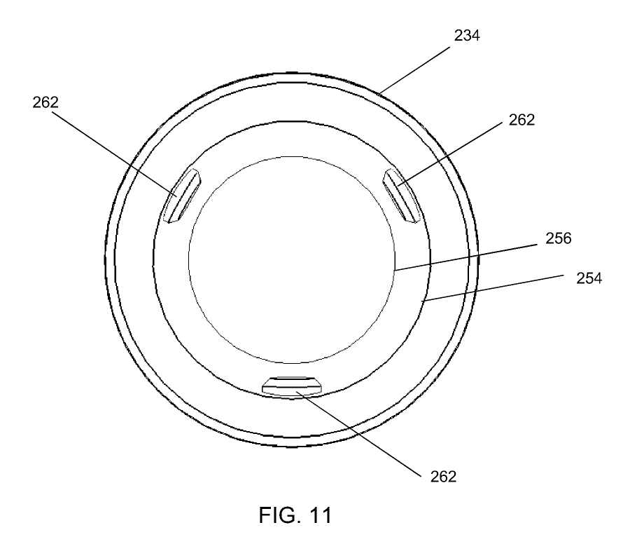

The fixation device 200 can include a barrel 234 with an exterior surface 236. The barrel 234 can include one or more retention structures 240. In the illustrate embodiment, the fixation device can include three retention structures 240. The number of retention structures 240 can correspond to the number of driving sections 228. The barrel 234 can include a taper 242 and a head 244. FIG. 11 is a view of the barrel 234 of the fixation device 200. The barrel 234 can include a lumen 254, a stop 256, and a socket 260 (not shown). The barrel 234 can have similar features to the barrel 134.

The fixation device 200 can function in a similar manner to fixation device 100. The fixation device 200 can lengthen and shorten. The fixation device 200 can include one or more engagement members 262. In the illustrate embodiment, the fixation device can include three engagement members 262. The one or more engagement members 262 can engage one or more driving sections 228. The one or more engagement members 262 can be cylindrical pins. The one or more engagement members 262 can be rounded to increase the surface area between the one or more engagement members 262 and the driving sections 228. The one or more engagement members 262 can be disposed radially inward. The one or more engagement members 262 can be disposed perpendicular to the longitudinal axis 206 of the fixation device 200. The elongate member 210 and the barrel 234 can be designed to translate relative to each other. The elongate member 210 and the barrel 234 can be rotationally coupled.

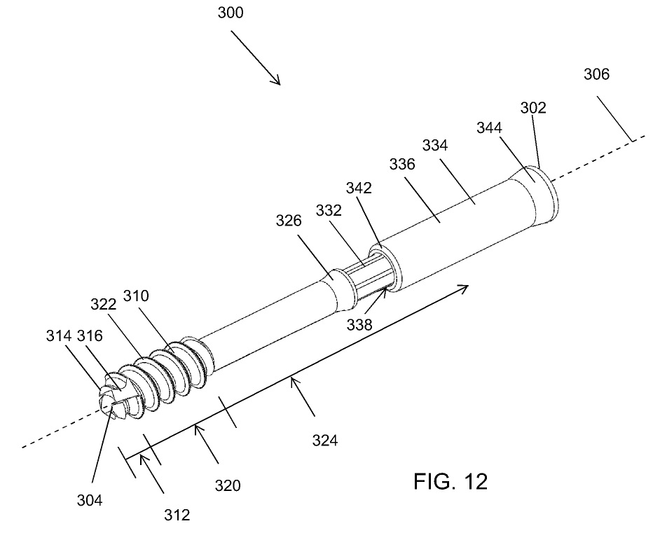

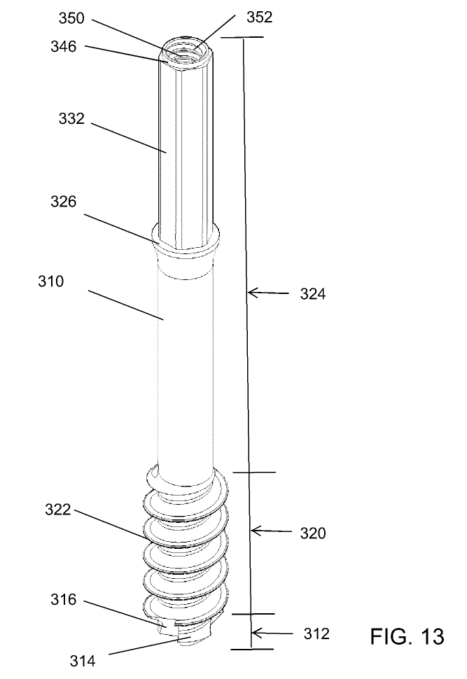

FIG. 12 shows an embodiment of a fixation device 300. The fixation device 300 can include any of the features described herein in relation to the fixation device 100 or the fixation device 200. The fixation device 300 can include a proximal end 302, a distal end 304, and a longitudinal axis 306. The fixation device 300 can include an elongate member 310. FIG. 13 is a view of the elongate member 310 of the fixation device 300. The elongate member 310 can include a first section 312 with one or more projections 314 and one or more cutting faces 316. The elongate member 310 can include a second section 320 with a thread 322.

The elongate member 310 can include a third section 324. The third section 324 can include a driving section 332. The driving section 332 can include an external surface. The external surface of the driving section 332 can be manufactured into the elongate member 310. The external surface of the driving section 332 can extend around the perimeter of the third section 324. The external surface of the driving section 332 can be a shaped design including one or more flat portions and/or one or more edges. The shape of the external surface of the driving section 332 can be selected from the group comprising torx, hex, 12-point flange, tri-wing, double-square, triple square, polydrive, spline drive, double hex, bristol, and pentalobe, etc. The shape of the external surface of the driving section 332 can be any non-round shape. In the illustrated embodiment, shape of the external surface of the driving section 332 is approximately a hex shape or a six-sided polygonal shape.

The elongate member 310 can include a shoulder 326. The elongate member 310 can include a head 346. The elongate member 310 can include a lumen 350 and a threaded portion 352 designed to interact with an inner screw 364, described herein. The elongate member 310 can have similar features to the elongate member 110 or the elongate member 210.

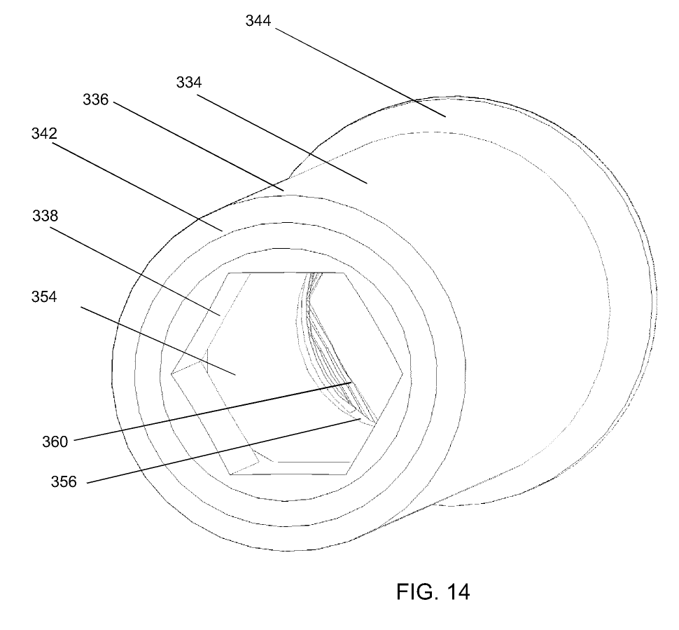

The fixation device 300 can include a barrel 334 with an exterior surface 336. FIG. 14 is a view of the barrel 334 of the fixation device 300. The barrel 334 can include a lumen 354, a stop 356, and a socket 360. The barrel 334 can include a head 344. The barrel 334 can have similar features to the barrel 134 or the barrel 234. The barrel 334 can include an interior surface 338 designed to engage the external surface of the driving section 332. The interior surface 338 of the barrel 334 can have a corresponding number of curved and/or flat sections as the exterior surface of the driving section 332. The interior surface 338 of the barrel 334 can form a socket. The shape of the interior surface 338 of the barrel 334 can be selected from the group comprising torx, hex, 12-point flange, tri-wing, double-square, triple square, polydrive, spline drive, double hex, bristol, and pentalobe, etc. The shape of the interior surface 338 of the barrel 334 can be any non-round shape. In the illustrated embodiment, shape of the interior surface 338 of the barrel 334 is approximately a hex shape or a six-sided polygonal shape.

The interior surface 338 of the barrel 334 can be manufactured into the barrel 334. The interior surface 338 of the barrel 334 can be integrally formed with the barrel 334. The interior surface 338 of the barrel 334 can be separately manufactured and inserted into the barrel 334. The interior surface 338 of the barrel 334 can be coupled to the barrel 334 through any technique known in the art (e.g., adhesive, o-rings, dove-tail configuration, thread, press-fit, friction fit, etc.). The barrel 334 can include a taper 342 and a head 344. The barrel 334 can include a lumen 354, a stop 356, and a socket 360 for imparting a rotational force on the barrel 334. The barrel 334 can have similar features to the barrel 134 or the barrel 234.

The fixation device 300 can function in a similar manner to fixation device 100 or the fixation device 200. The fixation device 300 can lengthen and shorten. The external surface of the driving section 332 of the elongate member 310 and the interior surface 338 of the barrel 334 can be designed to translate relative to each other. The elongate member 310 and the barrel 334 can be designed to translate relative to each other. The elongate member 310 and the barrel 334 can be rotationally coupled.

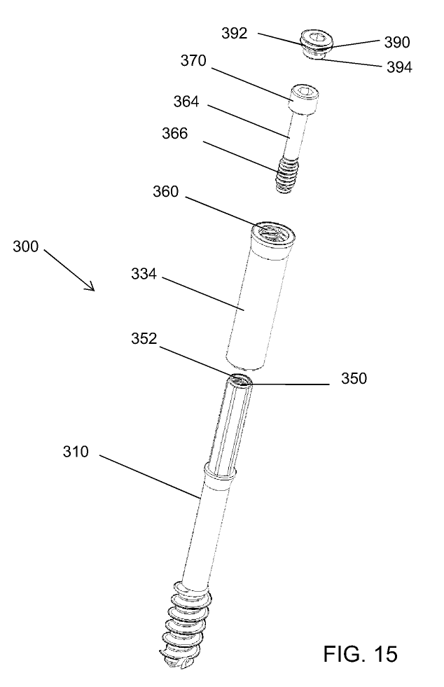

FIG. 15 is an exploded view of the fixation device 300. The fixation device 300 can include the elongate member 310. The fixation device 300 can include the barrel 334. The fixation device 300 can include the inner screw 364. The inner screw 364 can have similar features to the inner screw 164. The inner screw 364 can be designed to be inserted into the barrel 334. The inner screw 364 can be designed to be inserted into the elongate member 310.

The inner screw 364 can include a thread 366. The thread 366 can be designed to engage the threaded portion 352 of the lumen 350 of the elongate member 310. The inner screw 364 can include a head 370. The head 370 of the inner screw 364 can be designed to be inserted within the socket 360 of the barrel 334. The head 370 of the inner screw 364 can be designed to abut the stop 356, shown in FIG. 14. The stop 356 is designed to limit distal movement of the inner screw 364 within the barrel 334. In particular, the stop 356 is designed to limit distal movement of the head 370 of the inner screw 364 within the lumen 354 of the barrel 334. The head 370 can apply compressive force to the fixation device 300. The head 370 can apply a compressive force on the barrel 334.

The fixation device 300 can include one or more features to prevent back-out. The fixation device 300 can include one or more features to prevent loosening of the inner screw 364. The fixation device 300 can include a cap 390. The cap 390 can include a thread 392. The thread 392 can interact with the socket 360 of the barrel 334 or another threaded lumen of the barrel 334. The cap 390 can include a distal section 394. The distal section 394 can be designed to abut the head 370 of the inner screw 364. The cap 390 can form an interference fit or frictional fit with the barrel 134. The cap 390 can prevent withdrawal of the screw 364 once the screw 364 is inserted into the barrel 334. The inner screw 364 can be cannulated.

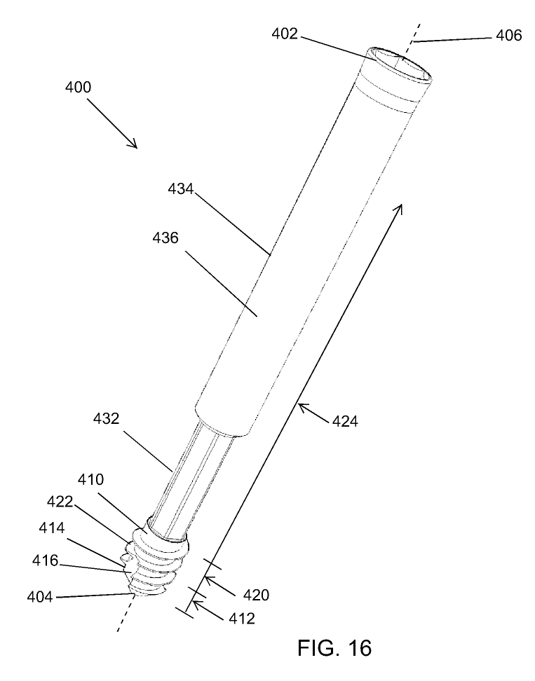

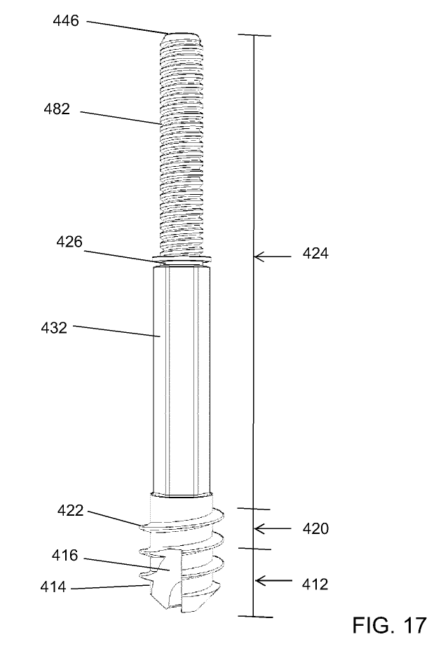

FIG. 16 shows an embodiment of a fixation device 400. The fixation device 400 can include any of the features described herein in relation to the fixation device 100, the fixation device 200, or the fixation device 300. The fixation device 400 can include a proximal end 402, a distal end 404, and a longitudinal axis 406. The fixation device 400 can include an elongate member 410. FIG. 17 is a view of the elongate member 410 of the fixation device 400. The elongate member 410 can include a first section 412 with one or more projections 414 and one or more cutting faces 416. The elongate member 410 can include a second section 420 with a thread 422.

The elongate member 410 can include a third section 424. The third section 424 can include a driving section 432. The driving section 432 can include an external surface. The external surface of the driving section 432 can be a shaped design including one or more flat portions and/or one or more edges. The shape of the external surface of the driving section 432 can be selected from the group comprising torx, hex, 12-point flange, tri-wing, double-square, triple square, polydrive, spline drive, double hex, bristol, and pentalobe, etc. The shape of the external surface of the driving section 332 can be any non-round shape. In the illustrated embodiment, shape of the external surface of the driving section 432 is approximately a hex shape or a six-sided polygonal shape. The elongate member 410 can include any of the features described herein in relation to the elongate member 110, the elongate member 210, or the elongate member 310.

The elongate member 410 can include a head 446. The elongate member 410 can include a shoulder 426. The elongate member 410 can include a thread 482 designed to interact with a nut 484, as described herein. The thread 482 can be disposed between the driving section 432 and the head 446. The thread 482 can include an external thread. In the illustrated embodiment, the thread 482 can include right-handed thread, but in some applications, left-handed thread can be used. The thread 482 can be the same handed thread as the thread 422 of the elongate member 410 (e.g., both right-handed thread).

Referring back to FIG. 16, the fixation device 400 can include a barrel 434 with an exterior surface 436. The barrel 434 can include a lumen 454 (not shown), a stop 356 (not shown), and a socket 460. The barrel 434 can include a head 444. The barrel 334 can have similar features to the barrel 134, the barrel 234 or the barrel 334. The barrel 434 can include an interior surface 438 designed to engage the external surface of the driving section 432. The interior surface 438 of the barrel 434 can have a corresponding number of curved and/or flat sections as the exterior surface of the driving section 432. The shape of the interior surface 438 of the barrel 434 can be selected from the group comprising torx, hex, 12-point flange, tri-wing, double-square, triple square, polydrive, spline drive, double hex, bristol, and pentalobe, etc. The shape of the interior surface 438 of the barrel 434 can be any non-round shape. The barrel 434 can include any of the features described herein in relation to the barrel 134, the barrel 234, or the barrel 334.

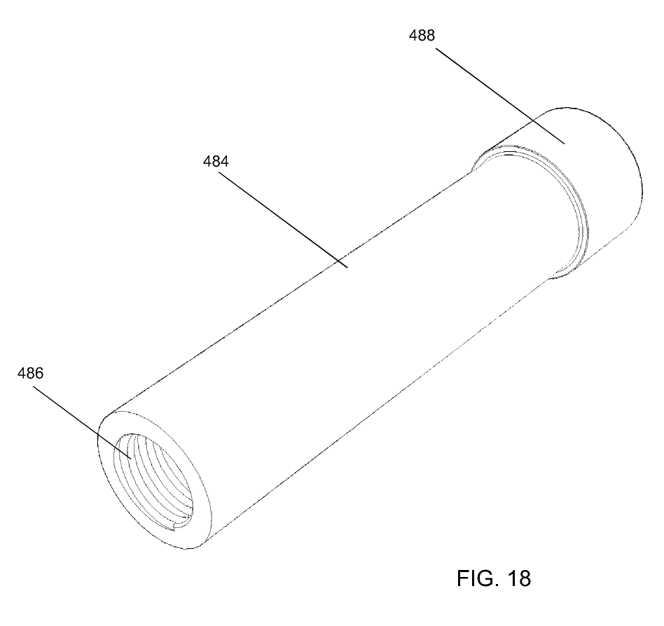

FIG. 18 is a view of the nut 284 of the fixation device 400. The elongate member 410 can include a first section 412 with one or more projections 414 and one or more cutting faces 416. The elongate member 410 can include a second section 420 with a thread 422.

The nut 484 can be designed to be inserted into the barrel 434. The nut 484 can be designed to be inserted over the thread 482 of the elongate member 410. The inner nut 484 can be designed to be inserted over the head 446 of the elongate member 410. The nut 484 can have a length sufficient to engage the elongate member 410 when the fixation device 400 is assembled.

The nut 484 can include a threaded portion 486. The threaded portion 486 can be an internal thread. The threaded portion 486 can be designed to engage the thread 482 of the elongate member 410. The nut 484 can include a head 488. The head 488 of the nut 484 can be designed to be inserted within the socket 460 of the barrel 434. The head 488 of the nut 484 can be designed to abut the stop 456. The stop 456 is designed to limit distal movement of the nut 484 within the barrel 434. In particular, the stop 456 is designed to limit distal movement of the head 488 of the nut 484 within the lumen 454 of the barrel 434. The nut 484 can have a length sufficient to allow the thread 486 of the nut 484 to engage the thread 482 of the elongate member 410 when the head 488 of the nut 484 abuts the stop 456.

The nut 484 can apply compressive force to the fixation device 400. The nut 484 can apply a compressive force on the barrel 434 to move the barrel 434 toward the distal end 404. The nut 484 can apply compressive force to the stop 456. The head 488 can apply compressive force to rigidly couple the elongate member 410 and the barrel 434. The nut 484 can be rotated to transmit a compressive force. In some embodiments, the nut 484 can be rotated in only one direction. In some embodiments, the nut 484 can be rotated in both directions. In some embodiments, the nut 484 can be rotated to shorten the fixation device 400. In some embodiments, the nut 484 can be rotated to lengthen the fixation device 400. As one example, rotation of the nut 484 in one direction can move the proximal end 402 toward the distal end 404. As another example, rotation of the nut 484 in the opposite direction can move the proximal end 402 away from the distal end 404.

The fixation device 400 is designed to permit translation between the barrel 434 and the elongate member 410 when the nut 484 is rotated. As one example, the barrel 434 can translate toward the elongate member 410 when the nut 484 is rotated. As one example, the interior surface 438 of the barrel 434 can translate relative to the driving section 432 when the nut 484 is rotated. In some embodiments, the elongate member 410 and the barrel 434 do not rotate when the nut 484 is rotated. The fixation device 400 is designed to limit rotation of the barrel 434 relative to the elongate member 410 when the nut 484 is rotated.

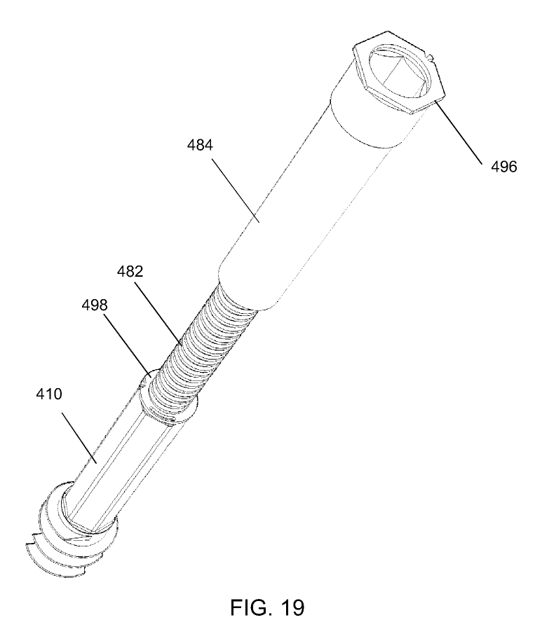

FIG. 19 is a view of components of the fixation device 400. The fixation device 400 can include the elongate member 410 and the nut 484. The nut 484 can be designed to be inserted over the thread 482 of the elongate member 410.

The fixation device 400 can include one or more features to prevent back-out of the nut 484 from the barrel 434. The fixation device 400 can include one or more features to prevent loosening of the nut 484. The fixation device 400 can include a cap 496. The cap 496 can be designed to engage a feature within the barrel 434. The cap 496 can include a projection. The cap 496 can include a shaped exterior surface. The cap 496 can include any non-round exterior surface. The cap 496 can interact with the socket 460 of the barrel 434 or another lumen of the barrel 434. The cap 496 can be designed to abut the head 488 of the nut 484. The cap 496 can form an interference fit or frictional fit with the barrel 434. The cap 496 can prevent withdrawal of the nut 484 once the nut 484 is inserted into the barrel 434. The cap 496 can be cannulated.

The fixation device 400 can include one or more features to prevent decoupling of one or more components of the fixation device 400. The fixation device 400 can include the stop 498. In some embodiments, the stop 498 can be designed to limit translation between the barrel 434 and the elongate member 410. In some embodiments, the stop 498 can be designed to limit distal movement of the head 446 of the elongate member 410 within the lumen 454 of the barrel 434. In some embodiments, the stop 498 can be designed to limit proximal movement of the shoulder 426 of the elongate member 410 within the lumen 454 of the barrel 434.

The size of the fixation devices 100, 200, 300, 400 can be based on the diameter of the thread 122, 222, 322, 422. The size of the fixation devices 100, 200, 300, 400 can be 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, between 2-4 mm, between 4-6 mm, between 6-8 mm, between 8-10 mm, etc. In some embodiments, the diameter of the exterior surface 136, 236, 336, 436 is equal or approximately equal to the diameter of the thread 122, 222, 322, 422. In some embodiments, the diameter of the head 144, 244, 344, 444 of the barrel 134, 234, 334, 324 is greater than the diameter of the thread 122, 222, 322, 422.

The method of use can include one or more of the following steps. In some methods of use, a pilot hole can be drilled across a bone. In some methods of use, a pilot hole can be drilled across two or more bone portions. In some methods of use, a pilot hole can be drilled across a fracture. In some methods of use, a pilot hole is drilled across a bone joint. In some methods of use, the pilot hole can be enlarged with a drill. In some methods of use, the pilot hole can be enlarged to the major diameter of the third section 124, 324, 334, 434. In some methods of use, the pilot hole can be enlarged to the diameter of the exterior surface 136, 236, 336, 436 of the barrel 134, 234, 334, 434. In some methods of use, the proximal surface of the bone can include a counter bore. The counter bore can be sized for the head 144, 244, 344, 444 of the barrel 134, 234, 334, 434. As described herein, the diameter of the head 144, 244, 344, 444 of the barrel 134, 234, 334, 434 is greater than the diameter of the thread 122, 222, 322, 422. The counter bore can allow the head 144, 244, 344, 444 of the barrel 134, 234, 334, 434 to be recessed in the bone.

In some methods of use, guide pin can be inserted into one or more bones. In some methods of use, the guide pin is inserted across a bone joint. In some methods of use, the guide pin is inserted across a fracture. In some methods of use, the guide pin is inserted across two bone portions. In some methods of use, the guide pin is inserted across two or more bones.

In some methods of use, the user is provided with one or more fixation devices 100, 200, 300, 400. The method can include the step of selecting a fixation device from a plurality of fixation devices 100, 200, 300, 400. The plurality of fixation devices 100, 200, 300, 400 can include two or more different fixation devices. The plurality of fixation devices 100, 200, 300, 400 can include two or more fixation devices of different sizes.

The fixation device 100, 200, 300, 400 can be inserted over the guide pin. As described herein, the fixation device 100, 200, 300, 400 is cannulated. The elongate member 110, 210, 310, 410 can include lumen 150, 250, 350, 450. The barrel 134, 234, 334, 434 can include lumen 154, 254, 354, 454. The fixation device 100, 200, 300, can include a passageway extending from the proximal end 102, 202, 302, 402 to the distal end 104, 204, 304, 404.

In some methods of use, the fixation device 100, 200, 300 is inserted over the guide pin without the inner screw 164, 264, 364. In some methods of use, the fixation device 100, 200, 300 is inserted over the guide pin without the inner screw 164, 264, 364 inserted in the barrel 134, 234, 334. In some methods of use, the fixation device 100, 200, 300 is inserted over the guide pin without the inner screw 164, 264, 364 engaging the elongate member 110, 210, 310. In some methods of use, the fixation device 100, 200, 300 is inserted over the guide pin without the inner screw 164, 264, 364 engaging the threaded portion 152, 252, 352 of the lumen 150, 250, 350 of the elongate member 110, 210, 310. In some methods of use, the fixation device 400 is inserted over the guide pin without the nut 484. In some methods of use, the fixation device 400 is inserted over the guide pin without the nut 484 inserted in the barrel 434. In some methods of use, the fixation device 400 is inserted over the guide pin without the nut 484 engaging the elongate member 410. In some methods of use, the fixation device 400 is inserted over the guide pin without the nut 464 engaging the thread 482 of the elongate member 410.

In alternative methods of use, the fixation device 100, 200, 300 is inserted over the guide pin with the inner screw 164. As described herein, the inner screw 164, 264, 364 can be cannulated. As described herein, the nut 484 can be cannulated. In some methods of use, the fixation device 100, 200, 300 is inserted over the guide pin with the inner screw 164, 264, 364 inserted in the barrel 134, 234, 334. In some methods of use, the fixation device 100, 200, 300 is inserted over the guide pin with the inner screw 164, 264, 364 engaging the elongate member 110, 210, 310. In some methods of use, the fixation device 100, 200, 300 is inserted over the guide pin with the inner screw 164, 264, 364 engaging the threaded portion 152, 252, 352 of the lumen 150, 250, 350 of the elongate member 110, 210, 310. In alternative methods of use, the fixation device 400 is inserted over the guide pin with the nut 484. In some methods of use, the fixation device 400 is inserted over the guide pin with the nut 484 inserted in the barrel 434. In some methods of use, the fixation device 400 is inserted over the guide pin with the nut 484 engaging the elongate member 410. In some methods of use, the fixation device 400 is inserted over the guide pin with the nut 464 engaging the thread 482 of the elongate member 410.

In some methods of use, the barrel 134, 234, 334, 434 can be rotated. As described herein, the barrel 134, 234, 334, 434 can include a socket 160, 260, 360, 460. The socket 160, 260, 360, 460 can be designed to accept a tool to rotate the barrel 134, 234, 334, 434. In some methods of use, the elongate member 110, 210, 310, 410 can rotate as the barrel 134, 234, 334, 434 is rotated. The barrel 134, 234, 334, 434 can be designed to transmits torque to the elongate member 110, 210, 310, 410.

The fixation device 100 can include one or more features to transmit torque. As described herein, the one or more engagement members 162 are designed to transmit torque from the barrel 134 to the elongate member 110. The one or more engagement members 162 can be coupled to the barrel 134. The elongate member 110 can include one or more driving sections 130. Each driving section 130 can include a flat surface 180 designed to interact with an engagement members 162. The one or more engagement members 162 can be arranged perpendicular to the longitudinal axis of the fixation device 100.

The fixation device 100 can include one or more features to prevent inadvertent rotation between the barrel 134 and the elongate member 110. As described herein, the one or more engagement members 162 are designed to rotationally couple the barrel 134 to the elongate member 110. The one or more engagement members 162 can be considered anti-rotation features. The fixation device 100 can include one or more features to prevent separation between the barrel 134 and the elongate member 110. As described herein, the one or more engagement members 162 are designed to limit release of the barrel 134 relative to the elongate member 110.

The fixation device 200 can include one or more features to transmit torque. As described herein, the one or more engagement members 262 are designed to transmit torque from the barrel 234 to the elongate member 210. The one or more engagement members 262 can be coupled to the barrel 234. The elongate member 110 can include one or more driving sections 228. The driving sections 228 can be designed to interact with the engagement members 262. Each driving section 228 can include a longitudinal groove designed to interact with an engagement member 262. The one or more engagement members 262 can be arranged parallel to the longitudinal axis of the fixation device 200.

The fixation device 200 can include one or more features to prevent inadvertent rotation between the barrel 234 and the elongate member 210. As described herein, the one or more engagement members 262 are designed to rotationally couple the barrel 234 to the elongate member 210. The one or more engagement members 262 can be considered anti-rotation features. The fixation device 200 can include one or more features to prevent separation between the barrel 234 and the elongate member 210. As described herein, the one or more engagement members 262 are designed to limit release of the barrel 234 relative to the elongate member 210.

The fixation device 300 can include one or more features to transmit torque. As described herein, the interior surface 338 is designed to transmit torque from the barrel 334 to the elongate member 310. The interior surface 338 can be integrally formed with the barrel 334. The elongate member 310 can include the driving section 332. The driving section 332 of the elongate member 310 can be designed to interact with the interior surface 338 of the barrel 334. The driving section 332 can be a non-round shape to transmit torque between the barrel 334 and the elongate member 310.

The fixation device 300 can include one or more features to prevent inadvertent rotation between the barrel 334 and the elongate member 310. As described herein, the interior surface 338 of the barrel 334 is designed to rotationally couple the barrel 334 to the elongate member 310. The interior surface 338 of the barrel 334 can be considered an anti-rotation feature. The fixation device 300 can include one or more features to prevent separation between the barrel 334 and the elongate member 310. In some embodiments, the interior surface 338 of the barrel 334 and/or the driving section 332 of the elongate member 310 are designed to limit release of the barrel 334 relative to the elongate member 310.

The fixation device 400 can include one or more features to transmit torque. As described herein, the interior surface 438 is designed to transmit torque from the barrel 434 to the elongate member 410. The interior surface 438 can be integrally formed with the barrel 434. The elongate member 410 can include the driving section 432. The driving section 432 of the elongate member 410 can be designed to interact with the interior surface 438 of the barrel 434. The driving section 432 can be a non-round shape to transmit torque between the barrel 434 and the elongate member 410.

The fixation device 400 can include one or more features to prevent inadvertent rotation between the barrel 434 and the elongate member 410. As described herein, the interior surface 438 of the barrel 434 is designed to rotationally couple the barrel 434 to the elongate member 410. The interior surface 438 of the barrel 434 can be considered an anti-rotation feature. The fixation device 400 can include one or more features to prevent separation between the barrel 434 and the elongate member 410. In some embodiments, the interior surface 438 of the barrel 434 and/or the driving section 432 of the elongate member 410 are designed to limit release of the barrel 434 relative to the elongate member 410.

In some methods of use, the elongate member 110, 210, 310, 410 can be driven into bone. In some methods of use, the elongate member 110, 210, 310, 410 can be driven into bone by rotation of the barrel 134, 234, 334, 434. In some methods of use, the elongate member 110, 210, 310, 410 can be driven into bone by rotation of the socket 160, 260, 360, 460 of the barrel 134, 234, 334, 343. In some methods of use, the elongate member 110, 210, 310, 410 can be driven into bone by rotation of the elongate member 110, 210, 310, 410. In some methods of use, the elongate member 110, 210, 310, 410 can be driven into bone by rotation of thread 122, 222, 322, 422. In some methods of use, the elongate member 110, 210, 310, 410 can be driven into bone by cutting bone with one or more projections 114, 214, 314, 314. In some methods of use, the elongate member 110, 210, 310, 410 can be driven into bone by cutting bone with one or more cutting faces 116, 216, 316, 416.

In some methods of use, the elongate member 110, 210, 310, 410 can be driven into bone until the head 144, 244, 344, 444 of the barrel 134, 234, 334, 434 abuts bone. In some methods of use, the elongate member 110, 210, 310, 410 can be driven into bone until the head 144, 244, 344, 444 of the barrel 134, 234, 334, 434 abuts the proximal bone surface. In some methods of use, the elongate member 110, 210, 310, 410 can be driven into bone until the head 144, 244, 344, 444 of the barrel 134, 234, 334, 434 abuts a counter bore surface in the bone.

In some methods of use, the fixation device 100, 200, 300, 400 is driven into bone in the second position. The second position can be the position of the shortest total length of the fixation device 100, 200, 300, 400. In some methods of use, the fixation device 100, 200, 300, 400 is driven into bone in a shortened position. The shortened position can be any position except the first position. The first position can be the position of the longest total length of the fixation device 100, 200, 300, 400.

In some methods of use, the barrel 134, 234, 334, 434 is rotated after the head 144, 244, 344, 444 of the barrel 134, 234, 334, 434 abuts bone. In some methods of use, the barrel 134, 234, 334, 434 is rotated after the head 144, 244, 344, 444 of the barrel 134, 234, 334, 434 abuts the proximal bone surface. In some methods of use, the barrel 134, 234, 334, 434 is rotated after the head 144, 244, 344, 444 of the barrel 134, 234, 334, 434 abuts the counter bore of the bone. The barrel 134, 224, 334, 434 can rotate without translating relative to the bone. The barrel 134, 224, 334, 434 can rotate in place.

In some methods of use, the elongate member 110, 210, 310, 410 can telescope relative to the barrel 134, 234, 334, 434. In some methods of use, the elongate member 110, 210, 310, 410 can telescope relative to the barrel 134, 234, 334, 434 after the head 144, 244, 344, 444 of the barrel 134, 234, 334, 434 abuts bone. The elongate member 110, 210, 310, 410 can be driven further into bone after the head 144, 244, 344, 444 of the barrel 134, 234, 334, 434 abuts bone. The elongate member 110, 210, 310, 410 can be driven further away from the proximal bone surface after the head 144, 244, 344, 444 of the barrel 134, 234, 334, 434 abuts bone.

In some methods of use, the fixation device 100, 200, 300, 400 can lengthen relative to the barrel 134, 234, 334, 434 after the head 144, 244, 344, 444 of the barrel 134, 234, 334, 434 abuts bone. In some methods of use, the fixation device 100, 200, 300, 400 lengthens to the first position. The first position can be the position of the longest total length of the fixation device 100, 200, 300, 400. In some methods of use, the fixation device 100, 200, 300, 400 lengthens beyond the shortened position.

In some methods of use, the fixation device 100, 200, 300, 400 lengthens until the position of the elongate member 110, 210, 310, 410 is optimized. In some methods of use, the fixation device 100, 200, 300, 400 is lengthened to extend across a bone joint. In some methods of use, the fixation device 100, 200, 300, 400 is lengthened to extend across a fracture. In some methods of use, the fixation device 100, 200, 300, 400 is lengthened to extend across two or more bones.

In some methods of use, the inner screw 164, 264, 364 is inserted into the fixation device 100, 200, 300 after the elongate member 110, 210, 310 is positioned within the bone. In some methods of use, the inner screw 164, 264, 364 is inserted into the fixation device 100, 200, 300 after the position of the elongate member 110, 210, 310 is optimized. As described herein, the inner screw 164, 264, 364 can be inserted into the barrel 134, 234, 334 of the fixation device 100, 200, 300. In some methods of use, the inner screw 164, 264, 364 engages the threaded portion 152 of the lumen 150 of the elongate member 110, 210, 310. In some methods of use, the inner screw 164, 264, 364 abuts the stop 156, 256, 346 of the barrel 134, 234, 334. In some methods of use, the inner screw 164, 264, 364 abuts the stop 156, 256, 356 of the barrel 134, 234, 334 to lock the inner screw 164, 264, 364 within the barrel 134, 234, 334. In some methods of use, the inner screw 164, 264, 364 abuts the stop 156, 256, 356 of the barrel 134, 234, 334 to prevent the inner screw 164, 264, 364 from being withdrawn from the barrel 134, 234, 334. In some methods of use, the inner screw 164, 264, 364 is retained within the barrel 134, 234, 334. In some methods of use, the head 170, 270, 370 of the screw 164, 264, 364 includes one or more features to prevent removal from the barrel 134, 234, 334. In some methods of use, the head 170, 270, 370 of the screw 164, 264, 364 forms a press-fit or friction fit with the barrel 134, 234, 334.

In some methods of use, the inner screw 164, 264, 364 compresses the fixation device 100, 200, 300. In some methods of use, the inner screw 164, 264, 364 shortens the fixation device 100, 200, 300. In some methods of use, the inner screw 164, 264, 364 increases the rigidity of the fixation device 100, 200, 300. In some methods of use, the inner screw 164, 264, 364 compresses the barrel 134, 234, 334 toward the elongate member 110, 210, 310. In some methods of use, the inner screw 164, 264, 364 compresses the elongate member 110, 210, 310 toward the barrel 134, 234, 334. In some methods of use, the inner screw 164, 264, 364 compresses two or more bones. In some methods of use, the inner screw 164, 264, 364 compresses a bone joint.

In some methods of use, the nut 484 is inserted into the fixation device 400 after the elongate member 410 is positioned within the bone. In some methods of use, the nut 484 is inserted into the fixation device 400 after the position of the elongate member 410 is optimized. As described herein, the nut 484 can be inserted into the barrel 434 of the fixation device 400. In some methods of use, the nut 484 engages the thread 482 of the elongate member 410. In some methods of use, the nut 484 abuts the stop 456 of the barrel 434. In some methods of use, the nut 484 abuts the stop 456 of the barrel 434 to lock the nut 484 within the barrel 434. In some methods of use, the nut 484 abuts the stop 456 of the barrel 434 to prevent the nut 484 from being withdrawn from the barrel 434. In some methods of use, the nut 484 is retained within the barrel 434. In some methods of use, the fixation device 400 includes one or more features to prevent removal of the nut 484 from the barrel 434. In some methods of use, the cap 496 forms a press-fit or friction fit with the barrel 434.

In some methods of use, the nut 484 compresses the fixation device 400. In some methods of use, the nut 484 shortens the fixation device 400. In some methods of use, the nut 484 increases the rigidity of the fixation device 400. In some methods of use, the inner nut 484 compresses the barrel 434 toward the elongate member 410. In some methods of use, the nut 484 pulls the elongate member 410 toward the barrel 434. In some methods of use, rotation of the nut 484 draws the elongate member 410 into the barrel 434. In some methods of use, the nut 484 compresses two or more bones. In some methods of use, the nut 484 compresses a bone joint.

The method of manufacturing can include one or more of the following steps. The method can include the step of providing a barrel comprising a lumen and a retention structure. The method can include the step of inserting an elongate member through the lumen of the barrel. The method can include the step of inserting an engagement member in the retention structure, wherein the barrel is configured to transmit torque to the elongate member after inserting the engagement member.

In some embodiments, the elongate member and the barrel are configured to telescope along a longitudinal axis after inserting the engagement member. In some embodiments, the elongate member and the barrel are configured to translate a fixed distance after inserting the engagement member. In some embodiments, the engagement member comprises a cylindrical pin. In some embodiments, the retention structure comprises a cylindrical slot. In some embodiments, the engagement member translates along a flat surface of the elongate member. In some embodiments, the engagement member translates along a groove of the elongate member. In some embodiments, the engagement member is configured to extend through the barrel in a direction perpendicular to the longitudinal axis. In some embodiments, the engagement member is configured to extend through the barrel in a direction parallel to the longitudinal axis.

1. A fixation device comprising:
an elongate member;
a barrel, wherein the elongate member and the barrel are configured to telescope along a longitudinal axis, wherein the barrel is configured to transmit torque to the elongate member; and
an inner screw configured to be inserted into the barrel and engage the elongate member.

2. The fixation device of claim 1, wherein the inner screw is configured to translate the barrel toward the elongate member.

3. The fixation device of claim 2, wherein at least a portion of the inner screw is threaded.

4. The fixation device of claim 3, wherein at least a portion of the elongate member is threaded.

5. The fixation device of claim 1, wherein the elongate member comprises a lumen.

6. The fixation device of claim 5, wherein at least a portion of the lumen of the elongate member is threaded.

7. The fixation device of claim 6, wherein the barrel comprises a lumen.

8. The fixation device of claim 7, wherein at least a portion of the elongate member is configured to translate within the lumen of the barrel.

9. The fixation device of claim 8, wherein the fixation device is cannulated.

10. The fixation device of claim 1, further comprising one or more engagement members configured to engage a surface of the elongate member to transmit torque from the barrel to the elongate member.

11. The fixation device of claim 10, wherein the one or more engagement members comprises one or more cylindrical pins.

12. The fixation device of claim 11, wherein the surface comprises a flat surface.

13. The fixation device of claim 12, wherein the one or more engagement members are configured to extend through the barrel in a direction perpendicular to the longitudinal axis.

14. The fixation device of claim 10, wherein the surface comprises a groove.

15. The fixation device of claim 14, wherein the one or more engagement members are configured to extend through the barrel in a direction parallel to the longitudinal axis.

16. The fixation device of claim 10, wherein the one or more engagement members is a non-round internal surface of the barrel.

17. A method of use of a fixation device comprising:
inserting a fixation device into a bone, the fixation device comprising an elongate member and a barrel, wherein the elongate member and the barrel are coupled;
rotating the barrel to telescope the elongate member; and
inserting an inner screw into the fixation device.

18. The method of claim 17, wherein inserting the fixation device into bone comprises inserting the fixation device without the inner screw inserted into the fixation device.

19. The method of claim 17, wherein the elongate member and the barrel are coupled to allow translation between the elongate member and the barrel.

20. A method of use of a fixation device comprising:
inserting a fixation device into a bone, the fixation device comprising an elongate member and a barrel, wherein the elongate member and the barrel are coupled;
rotating the barrel to telescope the elongate member; and
inserting a nut into the barrel.
